(19) United States
(12) Patent Application Publication
(10) Pub. No.: US 2004/0197310 A1
(43) Pub. Date: Oct. 7, 2004

(54) Compositions and methods for using umbilical cord progenitor cells in the treatment of myocardial infarction

(76) Inventors: Paul R. Sanberg, Spring Hill, FL (US); Robert Henning, Tampa, FL (US); Juan Sanchez-Ramos, Tampa, FL (US); Alison Willing, Tampa, FL (US)

Correspondence Address: SUTHERLAND ASBILL & BRENNAN LLP, 999 PEACHTREE STREET, N.E., ATLANTA, GA, 30309, US

(21) Appl. No.: 10/777,425

(22) Filed: Feb. 12, 2004

Related U.S. Application Data
(60) Provisional application No. 60/319,942, filed on Feb. 12, 2003.

(51) Int. Cl. A61K 45/00

(52) U.S. Cl. 424/93.7

(57) ABSTRACT

The present invention provides compositions and methods for treating circulatory disorders, for treating myocardial infarctions, for producing cardiac muscle cells, and for treating injured tissue in an individual. More particularly, the present invention provides methods of treating circulatory disorders by administering an effective amount of a composition comprising an umbilical cord blood cell. In one embodiment, the circulatory disorder is myocardial infarction.

### CROSS REFERENCE TO RELATED APPLICATIONS

The present invention claims priority to U.S. provisional patent application Serial No. 60/319,542, filed Feb. 12, 2003, the entire contents of which are hereby incorporated by reference.

### BACKGROUND OF THE INVENTION

1. Field of the Invention

The field of this invention is the treatment of circulatory disorders using stem cells. More specifically, a HUCB cell is administered to an individual in need of treatment in order to repair damage to the circulatory system.

2. Background Art

Each year, one million Americans experience an acute myocardial infarction and approximately 500,000 die from complications of myocardial infarction (American Heart Association 1999 Heart and Stroke Statistical Update). Infarct size is a critical determinant of prognosis, since it directly determines the degree of impaired heart pump function and the magnitude of heart dilation.

In order to limit myocardial infarction size and minimize or prevent heart failure, researchers have recently begun to transplant cells into infarcted hearts. Embryonic cells, fetal/neonatal and adult cardiac muscle cells, atrial tumor cells, skeletal muscle cells, and bone marrow cells have been transplanted into damaged hearts to improve heart function. However, each cell type has certain advantages but also certain limitations.

Multipotential human and animal cells can be derived from the inner cell mass of the blastocyst and also from embryos, and have the capacity to differentiate into cells from all three primary germ layers (Kehat et al., 2001 J. Clin. Invest. 108:407-414; Etzion et al., 2001 J. Mol. Cell Cardiol. 33:1321-1330; Saki et al., 1999 Ann. Thorac. Surg. 68:2074-2081; Maltsev et al., 1993 Mech. Dev. 44:41-50). Embryonic stem cells (ES cells) injected into hearts express myocyte actin, myosin heavy chain and troponin I proteins and form intercalated disks, sinusnodal and atrial cell types, and induce new blood vessel formation in the host ventricle (Van Meter et al., 1995 J. Thoracic Cardiovasc. Surg. 110(5):1442-14482; Maltsev et al., 1993 Mech. Dev. 44:41-50; Min et al., 2002 J Applied Physiology 92:288-296; Soonpaa et al., 1994 Science 264:98-101; Koh et al., 1995 J. Clin. Inves. 96:2034-2042). In addition, these cells attenuate infarct thinning, LV dilation and myocardial dysfunction and persist for at least two months (Etzion et al., 2001 J. Mol. Cell Cardiol. 33:1321-1330; Saki et al., 1999 Ann. Thorac. Surg. 68:2074-2081). Ethical issues governing the procurement and use of human ES cells for therapeutic purposes have significantly limited the availability and use of these cells. In addition, human embryonic stem cells to date are typically isolated and/or maintained on mouse feeder cells in culture, which raises concerns about transmission of rodent prions and viruses to humans. Moreover, long-term cultures of human embryonic cells may result in genetic mutations that limit their usefulness (Amit et al., 2000 Dev. Biol. 227:271-278). These issues have spurred researches to pursue the use of skeletal muscle cells and bone marrow cells as alternatives for cardiomyoplasty.

Approximately 4% to 8% of mammalian skeletal muscle cells are skeletal myoblasts, which are capable of cellular division and muscle repair (Campion, 1984 Int. Rev. Cytol. 87:225-251). Skeletal myoblasts have been injected directly into infarcted heart or injected into the coronary arteries for myocardial implantation (Murry et al., 1996 J. Clin. Invest. 98:2512-2523; Scorsin et al., 2000 J Thorac. Cardiovasc. Surg. 119:1169-1175; Suzuki et al., 2001 Circ. 104(Supp1):I213-I217). These cells can replicate in the myocardium for approximately 7 days, form multinucleated myotubes, differentiate into mature myofibers, and can contract when externally stimulated (Scorsin et al., 2000 J Thorac. Cardiovasc. Surg. 119:1169-1175; Suzuki et al., 2001 Circ. 104(Supp1):I213-I217). Approximately 20-30% of myofibers develop characteristics of slow twitch muscle as the cells mature. However, the mature cells demonstrate the histological features of well-differentiated skeletal muscle, and not cardiac muscle (Ghostine et al., 2002 Circ. 106:I131-I137). Skeletal myoblast transplantation into infarcted hearts prevents significant deterioration in left ventricular ejection fraction and reportedly limits the amount of infarction fibrous tissue, although this may also be due to fibroblast secretion of metalloproteinases that may be transplanted with the myoblasts (Suzuki et al., 2001 Circ. 104(Supp1):I213-I217; Ghostine et al., 2002 Circ. 106:I131-I137; Rajnoch et al., 2001 J. Thor. Cardiovasc. Surg. 121:871-878). The new muscle may persist for 6 to 12 months (Ghostine et al., 2002 Circ. 106:I131-I137) and is more resistant to ischemic injury than cardiac muscle.

The number of skeletal myoblasts present in skeletal muscles, however, decreases with age. Consequently, as much as 10 grams of skeletal muscle are necessary for myoblast isolation for transplantation into hearts of large animals or man (Ghostine et al., 2002 Circ. 106:I131-I137). Cell culture is mandatory to obtain adequate numbers of autologus or allogenic myoblasts for successful transplantation and usually requires ≧12 days (Pouzet et al., 2000 Circ. 102;III210-III215). Moreover, cell cultures often contain fibroblasts despite preplating, centrifugation of cells, and Percoll sedimentation. Unfortunately skeletal muscle communication with host cardiomyocytes via gap junction proteins is poor and significantly deteriorates over time (Murry et al., 1996 J. Clin. Invest. 98:2512-2523; Pouzet et al., 2000 Circ. 102;III210-III215). In addition, the skeletal muscle cell transplants do not consistently express cardiac specific proteins, fail to reconstitute healthy myocardium, and are often insulated from the myocardium by scar tissue. Moreover, immunosuppressive therapy is necessary for allogenic skeletal muscle transplantation. At the present time, the primary role of skeletal muscle transplantation appears to be as a scaffold that limits myocardial infarction scar expansion.

Recently investigators have recognized that bone marrow, which contains both mesenchymal and hematopoietic progenitor cells, have the capacity to colonize different tissues, proliferate, and transdifferentiate into cell lineages of the host organ. Mesenchymal bone marrow progenitor cells can serve as precursors for muscle and hematopoietic progenitor cells and can serve as precursors to endothelial cells (Liechty et al., 2000 Nature Medicine 6(11):1282-1286). Bone marrow mesenchymal cells express class I human leukocyte antigens but do not express class II antigens, which significantly limits immune rejection (Pittenger et al., 1999 Science 284:143-147).

Bone marrow mesenchymal cells (MSC) implanted into the left ventricle after myocardial infarction (Shake et al., 2002 Ann Thorac. Surg. 73:1919-1926; Strauer et al., 2002 Circ. 106:1913-1918) can persist at the site of myocardial implantation for as long as 6 months. MSCs can express the myocardial proteins α-actinin, tropomyosin, troponin T, myosin heavy chain and phospholamban (Shake et al., 2002 Ann Thorac. Surg. 73:1919-1926; Orlic et al., 2001 Nature 410:70-705). When cultured with 5-azacytidine, transplanted bone marrow cells may also induce angiogenesis in the infarction scar (Tomita et al., 1999 Circ. 110:II247-II256). Enriched hematopoietic bone marrow progenitor cells can contribute to 1 to 3% of endothelial cells in newly formed blood capillaries in the “at risk” tissue adjacent to the myocardial infarction (Toma et al., 2002 Circ. 105:93-98; Prockop, 1997 Science 276:71-74). The differentiation process for bone marrow derived cells appears to require specific paracrine growth signals from host cardiomyocytes and electromechanical stimulation in the adult heart (Toma et al., 2002 Circ. 105:93-98). The bone marrow MSC-derived cells in infarcted ventricles as reported to date have an immature phenotype and it is unknown if these cells progress to mature myocytes.

Implantation of bone marrow MSC appears to attenuate ventricular infarction scar area and paradoxical systolic ventricular wall thinning (Shake et al., 2002 Ann Thorac. Surg. 73:1919-1926; Strauer et al., 2002 Circ. 106:1913-1918; Tomita et al., 1999 Circ. 110:II247-II256). In addition, infarcted hearts transplanted with bone marrow cells can demonstrate an augmentation in ventricular systolic wall thickening at 4 weeks with variable effects on left ventricular pressure & dP/dt (Liechty et al., 2000 Nature Medicine 6(11):1282-1286; Shake et al., 2002 Ann Thorac. Surg. 73:1919-1926; Strauer et al., 2002 Circ. 106:1913-1918; Orlic et al., 2001 Nature 410:70-705; Tomita et al., 1999 Circ. 110:II247-II256). The isolation of adequate numbers of bone marrow stem cells can require as much as a liter of bone marrow, and the cells expand poorly in culture (Prockop, 1997 Science 276:71-74.). Progenitor cell preparation and expansion in culture can require as long as 21 days (Tomita et al., 1999 Circ. 110:II247-II256). Moreover, bone marrow MSC when implanted in scar tissue can give rise to fibroblast-like cells and ultimately scar tissue (Wang et al., 2000 J Thorac. Cardiovasc. Surg. 120:999-1005.). The use of allogenic bone marrow cells usually requires the use of immunosuppressant drugs, which may contribute to the immature transplant cellular phenotype. Finally, the electromechanical properties of bone marrow mesencymal and hematopoietic stem cells transplanted into the heart have not been characterized and it is unknown whether successful long-term cardiac engraftment in damage myocardium can be achieved.

Each donor cell type for transplantation previously discussed is associated with significant ethical, biological, or technical limitations that must be overcome in order to be generally applicable to patients. For this reason, an alternative source of donor cells is sought.

Human umbilical cord blood cells (HUCBC) have recently been recognized as a rich source of hematopoietic and mesenchymal progenitor cells (Broxmeyer et al., 1992 Proc. Natl. Acad. Sci. USA 89:4109-4113). Previously, umbilical cord and placental blood were considered a waste product normally discarded at the birth of an infant. Cord blood cells are used as a source of transplantable stem and progenitor cells and as a source of marrow repopulating cells for the treatment of malignant diseases (i.e. acute lymphoid leukemia, acute myeloid leukemia, chronic myeloid leukemia, myelodysplastic syndrome, and nueroblastoma) and non-malignant diseases such as Fanconi's anemia and aplastic anemia (Kohli-Kumar et al., 1993 Br. J. Haematol. 85:419-422; Wagner et al., 1992 Blood 79;1874-1881; Lu et al., 1996 Crit. Rev. Oncol. Hematol 22:61-78; Lu et al., 1995 Cell Transplantation 4:493-503). A distinct advantage of HUCBC is the immature immunity of these cells that is very similar to fetal cells, which significantly reduces the risk for rejection by the host (Taylor & Bryson, 1985 J. Immunol. 134:1493-1497).

Human umbilical cord blood contains mesenchymal and hematopoietic progenitor cells, and endothelial cell precursors that can be expanded in tissue culture (Broxmeyer et al., 1992 Proc. Natl. Acad. Sci. USA 89:4109-4113; Kohli-Kumar et al., 1993 Br. J. Haematol. 85:419-422; Wagner et al., 1992 Blood 79;1874-1881; Lu et al., 1996 Crit. Rev. Oncol. Hematol 22:61-78; Lu et al., 1995 Cell Transplantation 4:493-503; Taylor & Bryson, 1985 J. Immunol. 134:1493-1497 Broxmeyer, 1995 Transfusion 35:694-702; Chen et al., 2001 Stroke 32:2682-2688; Nieda et al., 1997 Br. J. Haematology 98:775-777; Erices et al., 2000 Br. J. Haematology 109:235-242). The total content of hematopoietic progenitor cells in umbilical cord blood equals or exceeds bone marrow, and in addition, the highly proliferative hematopoietic cells are eightfold higher in HUCBC than in bone marrow and express hematopoietic markers such as CD14, CD34, and CD45 (Sanchez-Ramos et al., 2001 Exp. Neur. 171:109-115; Bicknese et al., 2002 Cell Transplantation 11:261-264; Lu et al., 1993 J. Exp Med. 178:2089-2096). HUCBC contain thrombopoietin, interleukin and can contain as many as 77.2 to 95% CD34 cells (Chen et al., 2001 Stroke 32:2682-2688; Nieda et al., 1997 Br. J. Haematology 98:775-777). Thrombopoietin causes proliferation of hematopoietic cells, suppresses apoptosis, and functions as a survival factor. HUCB cells with a mesenchymal phenotype express SH2, SH3, SH5, α-smooth muscle actin, MAB 1470, CD13, CD29, and CD49 (Erices et al., 2000 Br. J. Haematology 109:235-242). Cell cycle analysis indicates that >85% of the mesenchymal cells are in the GO/G1 phase, however, these cells are capable of proliferating with a population-doubling time of 48 hours (Erices et al., 2000 Br. J. Haematology 109:235-242). The immunotype and functional properties displayed by cord blood-derived mesenchymal cells closely resembles the characteristics assigned to bone marrow derived mesenchymal progenitor cells (Erices et al., 2000 Br. J. Haematology 109:235-242). HUCBC are available in unlimited quantities, can be cryoperserved for periods of 5 to 50 years with recovery of 60 to 100% of granulocyte-macrophage colony forming units, erythroid burst forming units, and granulocyte/erythrocyte/ macrophage/ megakaryocyte colony forming units (Broxmeyer et al., 1992 Proc. Natl. Acad. Sci. USA 89:4109-4113; Bicknese et al., 2002 Cell Transplantation 11:261-264; Zigova et al., 2003 Cell Transplantation 11:265-274).

HUCB cells have recently been used for the treatment of stroke and traumatic brain injury (Chen et al., 2001 Stroke 32:2682-2688; Sanchez-Ramos et al., 2001 Exp. Neur. 171:109-115; Zigova et al., 2003 Cell Transplantation 11:265-274). Ischemic brain tissue expresses chemotactic proteins, such as monocyte chemoattractant protein 1, and adhesion molecules such as intercellular adhesion molecule (ICAM) and vascular endothelial adhesion molecule that attract HUCBC (Chen et al., 2001 Stroke 32:2682-2688). HUCBC also express several adhesive related integrin and ICAM proteins that facilitate migration of these cells to ischemic tissue (Chen et al., 2001 Stroke 32:2682-2688).

In rats with stroke due to middle cerebral artery occlusion, HUCBC were administered intravenously at day one or at day seven after a cerebral artery occlusion and the treated rats documented significant improvement in functional recovery as determined by a 25% increase in the rotarod test and a 44% decrease in the Modified Neurological Severity Score (Chen et al., 2001 Stroke 32:2682-2688). The improvement is evident within 14 days of IV administration of the HUCBC. The majority of the HUCBC localize to the ischemic zone in the brain that borders on the necrotic zone when the brains are examined at 14 and 35 days after the HUCBC injection. Moreover, these HUCBC express proteins phenotypic of neural cells such as NeuN, microtubule-associated protein 2, astrocyte glial fibrillary acidic protein, and α-tubulin III (Chen et al., 2001 Stroke 32:2682-2688; Zigova et al., 2003 Cell Transplantation 11:265-274). It has also been documented that intravenous HUCBC reduces neurological deficits in rats subjected to traumatic brain injuries and produces a 20% improvement in rotarod test scores and a 55% decrease in neurological severity scores. HUCBC migrating to the brain in this study express neuronal NeuN and MAP-2 and the astrocytic marker GFAP and also integrate into the vascular walls within the boundary zone of the injured area. In two other separate studies, HUCBC treated in culture with retinoic acid and nerve growth factor, or basic fibroblast growth factor and human epidermal growth factor, change phenotype and express neuronal and glial marker proteins such as neurite outgrowth extension protein which enhances axonal growth, glypican-4, neuronal pentraxin II, neuronal PAS1, neuronal growth-associated protein 43, α-tubulin III and glial fibrillary acidic protein (Sanchez-Ramos et al., 2001 Exp. Neur. 171:109-115; Bicknese et al., 2002 Cell Transplantation 11:261-264). To date, no studies have been published on progenitor HUCBC treatment for acute myocardial infarction.

Because of the difficulty in effectively treating patients with circulatory disorders, especially using cell-based therapies, there is a need in the art for methods and compositions to enhance the treatment of modalities.

### SUMMARY OF THE INVENTION

It is an object of the present invention to overcome, or at least alleviate, one or more of the difficulties or deficiencies associated with the prior art. In that regard, the present invention provides methods and compositions to repair damage to the circulatory system of an individual in need thereof by administering HUCB cells.

In that regard, the present invention fulfills in part the need to identify new, unique methods for treating circulatory damage.

In one embodiment, the method of treating a circulatory disorder comprises administering an effective amount of a composition comprising an umbilical cord blood cell to an individual with a circulatory disorder. In a further embodiment, the invention provides a method for treating myocardial infarction, comprising administering a composition comprising a human umbilical cord blood cell to an individual having a myocardial infarction in an effective amount sufficient to produce cardiac muscle cells in the heart of the individual, wherein the umbilical cord blood cell differentiates into a cardiac muscle cell. The invention further encompasses a method of producing a cardiac muscle cell, comprising administering an effective amount of a composition comprising a human umbilical cord blood cell to an individual in need of treatment, wherein the human umbilical cord blood cell differentiates into a cardiac muscle cell.

The invention further provides for a method of treating an injured tissue in an individual comprising: (a) determining a site of tissue injury in the individual; and (b) administering an umbilical cord blood composition into and around the site of tissue injury, wherein the umbilical cord blood composition comprises a cell that differentiates into a cardiac muscle cell after administration. In a preferred embodiment, the tissue is cardiac muscle. In one embodiment, the umbilical cord blood composition comprises a mononuclear cell fraction isolated from human umbilical cord blood; plasma or fetal bovine serum, and DMSO. Preferably, the plasma is from an autologous source. In a further embodiment, the tissue injury is a myocardial infarction. In one embodiment, the umbilical cord blood composition is prepared by the steps comprising: (a) obtaining whole cord blood from a neonatal umbilical cord; (b) enriching the cord blood for mononuclear cells; and (c) resuspending the cord blood enriched for mononuclear cells with plasma or fetal bovine serum, and DMSO. In one alternate embodiment, the umbilical cord blood composition comprises at least about 6 million white blood cells per milliliter or approximately 6 million to approximately 9 million white blood cells per milliliter, wherein approximately 10-14% of the cells are granulocytes, and wherein approximately 1-4% of the cells are CD34⁺ cells.

In one embodiment of the above methods, the individual is a human and the umbilical cord blood cell is a human cell. It is preferred that the umbilical cord blood cell differentiates into a cardiac muscle cell. It is also contemplated that the circulatory disorder is selected from the group consisting of cardiomyopathy, myocardial infarction, and congenital heart disease. Preferably, the circulatory disorder is a myocardial infarction. The invention provides that the differentiation into a cardiac muscle cell treats myocardial infarction by reducing the size of the myocardial infarct. It is also contemplated that the differentiation into a cardiac muscle cell treats myocardial infarction by reducing the size of the scar resulting from the myocardial infarct.

The invention contemplates that umbilical cord blood cell is administered directly to heart tissue of an individual, or is administered systemically. It is preferred that the umbilical cord blood cell is administered within approximately 48 hours after the onset of the myocardial infarction. In a further embodiment, the umbilical cord blood cell is administered within approximately 6 to approximately 12 hours after the onset of the myocardial infarct.

### BRIEF DESCRIPTION OF THE DRAWINGS

FIG. 1 depicts a behavioral profile of stroke animals. Stroke animals treated with intra-arterial HUCB cells plus mannitol displayed significantly reduced motor asymmetry in the elevated body swing test at 3 days post-stroke in comparison to animals treated with controls (HUCB alone or intra-arterial (IA) vehicle alone).

FIG. 2 depicts a behavioral profile of stroke animals. Stroke animals treated with IA HUCB cells plus mannitol displayed decreased acquisition time on passive avoidance testing at 3 days post-stroke in comparison to animals treated with controls.

FIG. 3 depicts a behavioral profile of stroke animals. Stroke animals treated with IA HUCB cells plus mannitol displayed increased retention time on passive avoidance testing at 3 days post-stroke in comparison to animals treated with controls.

FIG. 4 depicts an analysis of infarct volume. IA HUCB cell grafts+mannitol significantly reduced the size of cerebral infarction compared to controls. However, pre-transplant exposure of HUCB cells to the neurotrophic factor antibody cocktail treatment, blocked the neuroprotective effects of HUCB cell grafts+mannitol.

FIG. 5 shows an analysis of neurotrophic factors in the brains of stroke animals that were not treated with HUCB cells. No significant elevations in the brain levels of neurotrophic factors were observed in animals that were treated with HUCB cells that had been previously treated with antibodies to neurotrophic factors.

FIG. 6 shows an analysis of neurotrophic factors. ELISA revealed that IA HUCB plus mannitol increased GDNF brain levels at 3 days post-stroke. These increases were blocked when the HUCB cells were treated with neurotrophic factor antibody.

### DETAILED DESCRIPTION OF THE INVENTION

The present invention provides methods and compositions to treat circulatory disorders. Preferably, the circulatory disorder is a myocardial infarction.

In one embodiment, the invention provides for a method of treating circulatory disorders comprising administering an effective amount of a composition comprising an umbilical cord blood cell to an individual with a circulatory disorder. In a further embodiment, the invention provides a method for treating myocardial infarction, comprising administering a composition comprising a human umbilical cord blood cell to an individual having a myocardial infarction in an effective amount sufficient to produce cardiac muscle cells in the heart of the individual, wherein the umbilical cord blood cell differentiates into a cardiac muscle cell. The invention further encompasses a method of producing a cardiac muscle cell, comprising administering an effective amount of a composition comprising a human umbilical cord blood cell to an individual in need of treatment, wherein the human umbilical cord blood cell differentiates into a cardiac muscle cell.

The invention further provides for a method of treating an injured tissue in an individual comprising: (a) determining a site of tissue injury in the individual; and (b) administering an umbilical cord blood composition into and around the site of tissue injury, wherein the umbilical cord blood composition comprises a cell that differentiates into a cardiac muscle cell after administration. In a preferred embodiment, the tissue is cardiac muscle. In one embodiment, the umbilical cord blood composition comprises a mononuclear cell fraction isolated from human umbilical cord blood; plasma or fetal bovine serum, and DMSO. Preferably, the plasma is from an autologous source. In a further embodiment, the tissue injury is a myocardial infarction. In one embodiment, the umbilical cord blood composition is prepared by the steps comprising: (a) obtaining whole cord blood from a neonatal umbilical cord; (b) enriching the cord blood for mononuclear cells; and (c) resuspending the cord blood enriched for mononuclear cells with plasma or fetal bovine serum, and DMSO. In a preferred embodiment, the umbilical cord blood composition comprises approximately 6 million to approximately 9 million white blood cells per milliliter. In an additional embodiment, the umbilical cord blood composition comprises at least about 6 million white blood cells per milliliter or approximately 6 million to approximately 9 million white blood cells per milliliter, wherein approximately 10-14% of the cells are granulocytes, and wherein approximately 1-4% of the cells are CD34⁺ cells.

In preferred embodiments of the above methods, the individual is a human and the umbilical cord blood cell is a human cell. It is preferred that the umbilical cord blood cell differentiates into a cardiac muscle cell. It is also contemplated that the circulatory disorder is selected from the group consisting of cardiomyopathy, myocardial infarction, and congenital heart disease. The invention provides that the differentiation into a cardiac muscle cell can treat myocardial infarction by reducing the size of the myocardial infarct. It is also contemplated that the differentiation into a cardiac muscle cell can treat myocardial infarction by reducing the size of the scar resulting from the myocardial infarct.

The invention contemplates that umbilical cord blood cell is administered directly to heart tissue, or is administered systemically. It is preferred that the umbilical cord blood cell is administered within approximately 48 hours after the onset of the myocardial infarction. In a further embodiment, the umbilical cord blood cell is administered within approximately 6 to approximately 12 hours after the onset of the myocardial infarct.

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5 th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized.

The present invention may be understood more readily by reference to the following detailed description of the preferred embodiments of the invention and the Examples included herein. However, before the present compounds, compositions, and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

The umbilical cord blood cells of the subject invention can be administered to patients, including veterinary (non-human animal) patients, to alleviate the symptoms of a variety of pathological conditions for which cell therapy is applicable. For example, the cells of the present invention can be administered to a patient to alleviate the symptoms of circulatory disorders such as cardiomyopathy, myocardial infarction, and congenital heart disease, neurological disorders such as stroke (e.g., cerebral ischemia, hypoxia-ischemia); neurodegenerative diseases, such as Huntington's disease, Alzheimer's disease, and Parkinson's disease; traumatic brain injury; spinal cord injury; epilepsy (e.g., seizures and convulsions); Tay Sach's disease (beta hexosaminidase deficiency); lysosomal storage disease; amyotrophic lateral sclerosis; meningitis; multiple sclerosis (MS) and other demyelinating diseases; neuropathic pain; Tourette's syndrome; ataxia, drug addition, such as alcoholism; drug tolerance; drug dependency; depression; anxiety; and schizophrenia. In a preferred embodiment of the present invention, the cells are administered to alleviate the symptoms of circulatory disorders. In a further embodiment, the cells are administered to treat myocardial infarction.

The present invention is also directed to a method of treating circulatory damage in the heart or peripheral vasculature which occurs as a consequence of genetic defect, physical injury, environmental insult or damage from a stroke, heart attack or cardiovascular disease (most often due to ischemia) in a patient, the method comprising administering (including transplanting), an effective number or amount of umbilical cord blood cells to the patient, wherein at least one of the umbilical cord blood cells differentiates into a cardiac muscle cell.

The present invention provides a novel method to treat circulatory disorders, preferably myocardial infarction, to treat injured tissue, or to produce a cardiac muscle cell by the administration of human umbilical cord blood cells to an individual in need thereof These cells readily differentiate into various cells of the body, such as cardiac muscle cells, to be used in transplantation into a target site on or within the patient's body, such as the peripheral vasculature and heart of a patient, e.g., for the treatment of circulatory disorders. Optionally, the HUBC cells can be administered to a patient in a multipotent state or differentiated to varying degrees.

In one aspect of the present invention, HUBC cells are provided, which are suitable for administering systemically or to a target anatomical site. The HUBC cells can be grafted into or nearby a patient's heart, for example, or may be administered systemically, such as, but not limited to, intra-arterial or intravenous administration.

Pharmaceutical compositions or umbilical cord blood compositions of the present invention preferably comprise HUBC cells in combination with plasma or fetal bovine serum, and DMSO. In one embodiment, the HUBC cells comprise a mononuclear cell fraction isolated from human umbilical cord blood. In another embodiment, the plasma is from an autologous source. In one embodiment, the umbilical cord composition is prepared by the steps comprising: (a) obtaining whole cord blood from a neonatal umbilical cord; (b) enriching the cord blood for mononuclear cells; and (c) resuspending the cord blood enriched for mononuclear cells with plasma or fetal bovine serum, and DMSO. In a further embodiment, the umbilical cord blood composition comprises at least about 6 million white blood cells per milliliter or approximately 6 million to approximately 9 million white blood cells per milliliter. In an additional embodiment, the umbilical cord blood composition comprises approximately 6 million to approximately 9 million white blood cells per milliliter, wherein approximately 10-14% of the cells are granulocytes, and wherein approximately 1-4% of the cells are CD34⁺ cells.

In one embodiment, the administration of a composition comprising an umbilical cord blood cell leads to a measurable decrease in the size of a myocardial infarct, or in the size of a scar resulting from a myocardial infarct in the heart of a patient when compared to the size of a myocardial infarct, or the size of a scar resulting from a myocardial infarct in the absence of HUCBC treatment, or in the absence of any treatment. In other embodiments, the administration of a composition comprising an umbilical cord blood cell leads to an improvement selected from the group consisting of an improvement in systolic function, an improvement in diastolic function, improved elasticity, improved muscle contractility, improved heart function, and combinations thereof.

The compositions and methods of the present invention may be used for the treatment of myocardial infarct. Preferably, the compositions and methods are utilized from immediately following myocardial infarct, up until approximately 28 days after myocardial infarct. Preferably, the compositions and methods are used within approximately 48 hours after myocardial infarct. More preferably, the compositions and methods are used within approximately 6 to approximately 12 hours after myocardial infarct.

The pharmaceutical compositions may further comprise a cardiac cell differentiation agent. Cardiac cell differentiation agents for use in the present invention are well known to those of ordinary skill in the art.

The pharmaceutical compositions may further comprise a pharmaceutically acceptable carrier.

The terms “patient” and “individual” are used herein to describe an animal, preferably a human, to whom treatment, including prophylactic treatment, with the cells according to the present invention, is provided. For treatment of those infections, conditions or disease states which are specific for a specific animal such as a human patient, the term patient refers to that specific animal. The term “donor” is used to describe an individual (animal, including a human) who or which donates umbilical cord blood or umbilical cord blood cells for use in a patient.

The term “umbilical cord blood” is used herein to refer to blood obtained from a neonate or fetus, most preferably a neonate and preferably refers to blood that is obtained from the umbilical cord or the placenta of newborns. Preferably, the umbilical cord blood is isolated from a human newborn. The use of umbilical cord blood as a source of mononuclear cells is advantageous because it can be obtained relatively easily and without trauma to the donor. In contrast, the collection of bone marrow cells from a donor is a traumatic experience. Umbilical cord blood cells can be used for autologous transplantation or allogenic transplantation, when and if needed. Umbilical cord blood is preferably obtained by direct drainage from the cord an/or by needle aspiration from the delivered placenta at the root and at distended veins. As used herein, the term “umbilical cord blood cells” refers to cells that are present within umbilical cord blood. In one embodiment, the umbilical cord blood cells are mononuclear cells that are further isolated from the umbilical cord blood using methods known to those of skill in the art. In a further embodiment, the umbilical cord blood cells may be further differentiated prior to administration to a patient. In a further embodiment, the umbilical cord blood cell is a mesenchymal cell or a hematopoietic cell. In one preferred embodiment, the umbilical cord blood cell is a mesenchymal cell.

The term “effective amount” is used herein to describe concentrations or amounts of components such as differentiation agents, umbilical cord blood cells, precursor or progenitor cells, specialized cells, such as cardiac muscle cells, and/or other agents which are effective for producing an intended result including differentiating stem and/or progenitor cells into specialized cells, such as cardiac muscle cells, or treating a circulatory disorder or other pathologic condition including damage to the cardiovascular system of a patient, such as a stroke, heart attack, or accident victim or for effecting a transplantation of those cells within the patient to be treated. Compositions according to the present invention may be used to effect a transplantation of the umbilical cord blood cells within the composition to produce a favorable change in the cardiovascular system, or in the disease or condition treated, whether that change is an improvement (such as stopping or reversing the degeneration of a disease or condition, reducing or reversing a block in cardiovascular function, or improving a cardiovascular function) or a complete cure of the disease or condition treated.

The terms “stem cell” or “progenitor cell” are used interchangeably herein to refer to umbilical cord blood-derived stem and progenitor cells. The terms stem cell and progenitor cell are known in the art (e.g., Stem Cells: Scientific Progress and Future Research Directions, report prepared by the National Institutes of Health, June, 2001). As used herein, the terms “cardiac muscle cells” and “cardiomyocyte” are used interchangeably and refer to cells having at least an indication of cardiac muscle or muscle phenotype, such as staining for one or more cardiac muscle or muscle markers or which will differentiate into cells exhibiting cardiac muscle or muscle markers. Examples of cardiac muscle markers which may be used to identify cardiac muscle cells according to the present invention include, for example, α-actinin, β-myosin heavy chain, and cardiac troponin I. Cardiac muscle phenotype may be indicated by the sarcomeric organization of contractile proteins in the cell. All of the above cells and their progeny are construed as cardiac muscle cells for the purpose of the present invention. As used herein, the term “endothelial cells” refers to cells having at least an indication of endothelial phenotype, such as staining for one or more endothelial markers or which will differentiate into cells exhibiting endothelial markers. Examples of endothelial markers which may be used to identify endothelial cells according to the present invention include, for example, the VEGF endothelial cell receptor Flt-1 involved in endothelial cell growth, Von Willebrand factor, and Factor VIII, which are present in endothelial cells but not HUCBC. All of the above cells and their progeny are construed as endothelial cells for the purpose of the present invention.

The term “administration” or “administering” is used throughout the specification to describe the process by which cells of the subject invention, such as umbilical cord blood cells obtained from umbilical cord blood, or more differentiated cells obtained therefrom, are delivered to a patient for therapeutic purposes. Cells of the subject invention be administered a number of ways including, but not limited to, parenteral (such term referring to intravenous and intra-arterial as well as other appropriate parenteral routes), intrathecal, intraventricular, intraparenchymal (including into the spinal cord, brainstem or motor cortex), intracisternal, intracranial, intrastriatal, and intranigral, among others which term allows cells of the subject invention to migrate to the ultimate target site where needed. In one embodiment, the cells are administered in proximity to the injured tissue, or administered directly to the heart tissue. Cells of the subject invention can be administered in the form of intact umbilical cord blood or a fraction thereof (such term including a mononuclear fraction thereof or a fraction of mononuclear cells, including a high concentration of stem or progenitor cells). The compositions according to the present invention may be used without treatment with a mobilization agent or differentiation agent (“untreated” i.e., without further treatment in order to promote differentiation of cells within the umbilical cord blood sample) or after treatment (“treated”) with a differentiation agent or other agent which causes certain stem and/or progenitor cells within the umbilical cord blood sample to differentiate into cells exhibiting a differentiated phenotype, such as a cardiac muscle phenotype.

The umbilical cord blood stem or progenitor cells can be administered systemically or to a target anatomical site, permitting the cells to differentiate in response to the physiological signals encountered by the cell (e.g., site-specific differentiation). Alternatively, the cells may undergo ex vivo differentiation prior to administration into a patient.

Administration will often depend upon the disease or condition treated and may preferably be via a parenteral route, for example, intravenously, or intra-arterially or by direct administration into the affected tissue in the heart. For example, in the case of myocardial infarct, a preferred route of administration will be a transplant directly into the injured tissue (which may be readily determined using MRI or other imaging techniques), or may be administered systemically. In the case of peripheral vascular disease, the preferred administration is intravenously or intra-arterially. In the case of lysosomal storage disease, the preferred route of administration is via an intravenous route or through the cerebrospinal fluid.

The terms “grafting” and “transplanting” and “graft” and “transplantation” are used throughout the specification synonymously to describe the process by which cells of the subject invention are delivered to the site where the cells are intended to exhibit a favorable effect, such as repairing damage to a patient's cardiovascular system, treating a cardiovascular disease or treating the effects of damage caused by stroke, cardiovascular disease, a heart attack or physical injury or trauma or genetic damage or environmental insult to the cardiovascular system, caused by, for example, an accident or other activity. Cells of the subject invention can also be delivered in a remote area of the body by any mode of administration as described above, relying on cellular migration to the appropriate area to effect transplantation.

The term “non-tumorigenic” refers to the fact that the cells do not give rise to a neoplasm or tumor. Stem and/or progenitor cells for use in the present invention are preferably free from neoplasia and cancer.

The term “differentiation agent” or “cardiac differentiation agent” is used throughout the specification to describe agents which may be added to cell culture (which term includes any cell culture medium which may be used to grow cardiac muscle cells according to the present invention) containing umbilical cord blood pluripotent or multipotent stem and/or progenitor cells which will induce the cells to a more differentiated phenotype, such as a cardiac muscle or muscle phenotype. Alternatively, a differentiation agent may be administered to the patient separately from the HUCB cells of the present invention. As used herein, the term “differentiate” refers to partially or terminal differentiation of a cell.

The term “cardiovascular disease” and “circulatory disorder” are used interchangeably, and are used herein to describe a disease or disorder which is caused by damage to the circulatory system and which damage can be reduced and/or alleviated through transplantation of HUCB cells according to the present invention to damaged areas of the heart and/or circulatory system of the patient. As used herein, the term “circulatory damage” is used to refer to injury to the circulatory system that may be caused be any of a number of diseases or disorders. Exemplary cardiovascular diseases which may be treated using the cells and methods according to the present invention include for example, myocardial infarct, cardiomyopathy, peripheral vascular disease, congenital heart disease, other genetic diseases, and injury or trauma caused by ischemia, accidents, environmental insult. In addition, the present invention may be used to reduce and/or eliminate the effects on the central nervous system of a heart attack in a patient, which is otherwise caused by lack of blood flow or ischemia to a site in the brain of said patient or which has occurred from physical injury to the brain and/or spinal cord.

The term “gene therapy” is used throughout the specification to describe the transfer and stable insertion of new genetic information into cells for the therapeutic treatment of diseases or disorders. The foreign gene is transferred into a cell that proliferates to spread the new gene throughout the cell population. Thus, umbilical cord blood cells, or progenitor cells are the targets of gene transfer either prior to differentiation or after differentiation to a neural cell phenotype. The umbilical cord blood stem or progenitor cells of the present invention can be genetically modified with a heterologous nucleotide sequence and an operably linked promoter that drives expression of the heterologous nucleotide sequence. The nucleotide sequence can encode various proteins or peptides of interest. The gene products produced by the genetically modified cells can be harvested in vitro or the cells can be used as vehicles for in vivo delivery of the gene products (i.e., gene therapy).

The following written description provides exemplary methodology and guidance for carrying out many of the varying aspects of the present invention.

### Molecular Biology Techniques

Standard molecular biology techniques known in the art and not specifically described are generally followed as in Sambrook et al., Molecular Cloning: A Laboratory Manual, Cold Springs Harbor Laboratory, N.Y. (1989, 1992), and in Ausubel et al., Current Protocols in Molecular Biology, John Wiley and Sons, Baltimore, Md. (1989). Polymerase chain reaction (PCR) is carried out generally as in PCR Protocols: A Guide to Methods and Applications, Academic Press, San Diego, Calif. (1990). Reactions and manipulations involving other nucleic acid techniques, unless stated otherwise, are performed as generally described in Sambrook et al., Molecular Cloning: A Laboratory Manual, Cold Springs Harbor Laboratory Press, and methodology as set forth in Unites States Patent Nos. 4,666,828; 4,683,202; 4,801,531; 5,192,659; and 5,272,057 and incorporated herein by reference. In situ PCR in combination with Flow Cytometry can be used for detection of cells containing specific DNA and mRNA sequences (see, for example, Testoni et al., Blood, 1996, 87:3822).

Standard methods in immunology known in the art and not specifically described are generally followed as in Stites et al. (Eds.), Basic And Clinical Immunology, 8^{th} Ed., Appleton & Lange, Norwalk, Conn. (1994); and Mishell and Shigi (Eds.), Selected Methods in Cellular Immunology, W. H. Freeman and Co., New York (1980).

### Immunoassays

In general, immunoassays are employed to assess a specimen such as for cell surface markers or the like. Immunocytochemical assays are well known to those skilled in the art. Both polyclonal and monoclonal antibodies can be used in the assays. Where appropriate other immunoassays, such as enzyme-linked immunosorbent assays (ELISAs) and radioimmunoassays (RIA), can be used as are known to those in the art. Available immunoassays are extensively described in the patent and scientific literature. See, for example, U.S. Pat. No. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771; and 5,281,521 as well as Sambrook et al., Molecular Cloning: A Laboratory Manual, Cold Springs Harbor, N.Y., 1989. Numerous other references also may be relied on for these teachings.

### Antibody Production

Antibodies may be monoclonal, polyclonal, or recombinant. Conveniently, the antibodies may be prepared against the immunogen or immunogenic portion thereof, for example, a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art as described generally in Harlow and Lane, Antibodies: A Laboratory Manual, Cold Spring Harbor Laboratory, Cold Springs Harbor, N.Y. (1988) and Borrebaeck, Antibody Engineering− A Practical Guide by W. H. Freeman and Co. (1992). Antibody fragments may also be prepared from the antibodies and include Fab and F(ab′)2 by methods known to those skilled in the art. For producing polyclonal antibodies a host, such as a rabbit or goat, is immunized with the immunogen or immunogenic fragment, generally with an adjuvant and, if necessary, coupled to a carrier; antibodies to the immunogen are collected from the serum. Further, the polyclonal antibody can be absorbed such that it is monospecific. That is, the serum can be exposed to related immunogens so that cross-reactive antibodies are removed from the serum rendering it monospecific.

For producing monoclonal antibodies, an appropriate donor is hyperimmunized with the immunogen, generally a mouse, and splenic antibody-producing cells are isolated. These cells are fused to immortal cells, such as myeloma cells, to provide a fused cell hybrid that is immortal and secretes the required antibody. The cells are then cultured, and the monoclonal antibodies harvested from the culture media.

For producing recombinant antibodies, messenger RNA from antibody-producing B-lymphocytes of animals or hybridoma is reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system. Antibody cDNA can also be obtained by screening pertinent expression libraries. The antibody can be bound to a solid support substrate or conjugated with a detectable moiety or be both bound and conjugated as is well known in the art. (For a general discussion of conjugation of fluorescent or enzymatic moieties see Johnstone & Thorpe, Immunochemistry in Practice, Blackwell Scientific Publications, Oxford, 1982). The binding of antibodies to a solid support substrate is also well known in the art. (see for a general discussion Harlow & Lane, Antibodies: A Laboratory Manual, Cold Spring Harbor Laboratory Publications, New York, 1988 and Borrebaeck, Antibody Engineering—A Practical Guide, W. H. Freeman and Co., 1992). The detectable moieties contemplated with the present invention can include, but are not limited to, fluorescent, metallic, enzymatic and radioactive markers. Examples include biotin, gold, ferritin, alkaline phosphates, galactosidase, peroxidase, urease, fluorescein, rhodamine, tritium, ¹⁴C, iodination and green fluorescent protein.

### Gene Therapy

Gene therapy as used herein refers to the transfer of genetic material (e.g., DNA or RNA) of interest into a host to treat or prevent a genetic or acquired disease or condition. The genetic material of interest encodes a product (e.g., a protein, polypeptide, and peptide, functional RNA, antisense) whose in vivo production is desired. For example, the genetic material of interest encodes a hormone, receptor, enzyme polypeptide or peptide of therapeutic value. Alternatively, the genetic material of interest encodes a suicide gene. For a review see “Gene Therapy” in Advances in Pharmacology, Academic Press, San Diego, Calif., 1997.

### Administration of Cells for Transplantation

The umbilical cord blood cells of the present invention can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art.

In the method of the present invention, the umbilical cord blood cells of the present invention can be administered in various ways as would be appropriate to implant in the central nervous system, including but not limited to parenteral, including intravenous and intraarterial administration, intrathecal administration, intraventricular administration, intraparenchymal, intracranial, intracisternal, intrastriatal, and intranigral administration. Optionally, the umbilical cord blood cells are administered in conjunction with an immunosuppressive agent.

Pharmaceutical compositions comprising effective amounts of umbilical cord blood cells are also contemplated by the present invention. These compositions comprise an effective number of cells, optionally, in combination with a pharmaceutically acceptable carrier, additive or excipient. In certain aspects of the present invention, cells are administered to the patient in need of a transplant in sterile saline. In other aspects of the present invention, the cells are administered in Hanks Balanced Salt Solution (HBSS) or Isolyte S, pH 7.4. Other approaches may also be used, including the use of serum free cellular media. In one embodiment, the cells are administered in plasma or fetal bovine serum, and DMSO. Systemic administration of the cells to the patient may be preferred in certain indications, whereas direct administration at the site of or in proximity to the diseased and/or damaged tissue may be preferred in other indications.

Pharmaceutical compositions according to the present invention preferably comprise an effective number within the range of about 1.0×10⁴ cells to about 5.0×10⁷ cells, more preferably about 1×10⁵ to about 10×10⁶ cells, even more preferably about 6×10⁶ to about 9×10⁶ cells generally in solution, optionally in combination with a pharmaceutically acceptable carrier, additive or excipient.

In one embodiment, the umbilical cord blood cells are administered with a differentiation agent. In one embodiment, the cells are combined with the differentiation agent to administration into the patient. In another embodiment, the cells are administered separately to the patient from the differentiation agent. Optionally, if the cells are administered separately from the differentiation agent, there is a temporal separation in the administration of the cells and the differentiation agent. The temporal separation may range from about less than a minute in time, to about hours or days in time. The determination of the optimal timing and order of administration is readily and routinely determined by one of ordinary skill in the art.

Throughout this application, various publications are referenced. The disclosures of all of these publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### EXAMPLES

### Example 1

Administration of HUCBC Decreases Damage of Acute Anterior Wall Infarction Methods

Acute myocardial infarction was induced in rats as follows. Male Sprague-Dawley rats, weighing 250-350 grams, were anesthetized with ketamine 30-90 mg/kg and xylazine 2-9 mg/kg, and maintained under surgical anesthesia. Each animal was intubated with PE190 tubing and mechanically ventilated with room air using a tidal volume of approximately 1.2 ml/100 g body weight and a rate of 60 strokes/min. A thoracotomy was performed through the left 4^{th} intercostal space. A pericardectomy was performed and the left anterior descending coronary artery was ligated near its origin with 4-0 silk suture (Johns & Olson, 1954 Ann Surgery 140:675-682). Collateral coronary circulation in the rat was negligible. Infarction was confirmed by anterior wall cyanosis, akinesis, and electrocardiographic changes of ST segment elevation. The chest wall was closed in two layers with absorbable polyglycolic suture and the skin closed with staples. Bupivacaine hydrochloride (0.1 mg/kg) was injected into the intercostal muscle at 3 sites around the incision to provide anesthesia. Thereafter, the animal recovered on a heating pad and was returned to its cage. Buprenorpine (0.1-0.5 mg/kg sq) was given the day following surgery for analgesia to rats that were experiencing discomfort. Cephalothin 40mg/kg/day and gentamycin 5 mg/kg ql2 hour were administered on day 1 then given q 24 for signs of infection. To date, no rat has acquired an infection.

Human umbilical blood progenitor cells were obtained as follows. Written consent to obtain umbilical cord blood was obtained from the mother prior to delivery. The USF IRB has reviewed/approved the consent form. Whole cord- blood was obtained from the umbilical cord following the birth of the child after the cord was clamped. Maternal blood was tested for HIV, hepatitis, syphilis, cytomegalic virus, and HTLV and the blood was rejected if any of these tests were positive. Sterile technique was used throughout the isolation procedures (Toma et al., 2002 Circulation 105:93-98; Gee, Bone Marrow Processing, a Practical Guide. CRC Press 1991).

The umbilical cord blood was spun at 400 g for 15 minutes and the plasma removed. Sterile Dulbecco Phosphate Buffered saline was mixed with the cord blood. The solution was then underlaid with sterile Lymphocyte Separation Medium (LSM) (Ficoll-Hypaque) and the entire solution was spun at 400 g for 30 minutes. After spinning, a distinct layer of mononuclear cells was visible at the interface of the plasma and Ficoll. The top plasma layer was removed with a pipette. The mononuclear cells were then removed and placed in a tube with RPMI in a dilution of 1:2. The contents were spun at 400 g for 15 minutes. The supernatant was carefully decanted without disturbing the pelleted cells. RPMI was again added and the solution was spun at 400 g for 10 minutes. The supernatant was decanted and the cells were mixed with a small quantity of RPMI. 0.5 ml of cells was removed with a sterile transfer pipette and the white blood cell count, CD34 determination, and cell viability determined by Trypan Blue technique. Cell viability was generally 85-95%. The remaining cells were spun at 400 g for 10 minutes. 0.5 ml of the supernatant was removed and placed in a pediatric microbiology blood culture tube. The remaining supernatant was removed and RPMI added to the cells. Autologous plasma or fetal bovine serum and DMSO were then added in a drop wise manner to the cord cells and the solution mixed. Aliquot cell suspensions were then placed in sterile cryovials and the samples placed in a controlled rate freezer. All cell samples were stored at −196° C. in liquid nitrogen vapor. Cell samples contained approximately 7.4 million white blood cells per millimeter, 11.6% granulocytes, and 1-4% CD34+ cells.

### Results

Preliminary studies were performed on 23 male Sprague Dawley rats that were matched for age and weight to determine whether HUCBC administration limits the damage of acute anterior wall infarction.

In this study, Group I consisted of 8 control rats with no interventions in order to determine whether any heart changes were due to increases in age and weight. Group 2 consisted of 15 rats with anterior wall ventricular infarctions from ligation of the left anterior descending coronary artery, and Group 3 consisted of 9 rats with anterior wall ventricular infarctions plus one million HUCBC that were circumferentially injected directly in the periphery (i.e. border ischemic zones) of the ventricular infarction one hour after acute infarction. In Group 2, only the transport medium (Isolyte), but no cells, was injected into the border ischemic zone of the left ventricle one hour after infarction. All the rats were monitored with echocardiograms performed prior to and at 1, 2, 3, and 4 months after the infarction. Immunosuppressive therapy was not given to any rat. One to 2 rats from each group were sacrificed at 1, 2, 3, and 4 months for immunohistochemical studies.

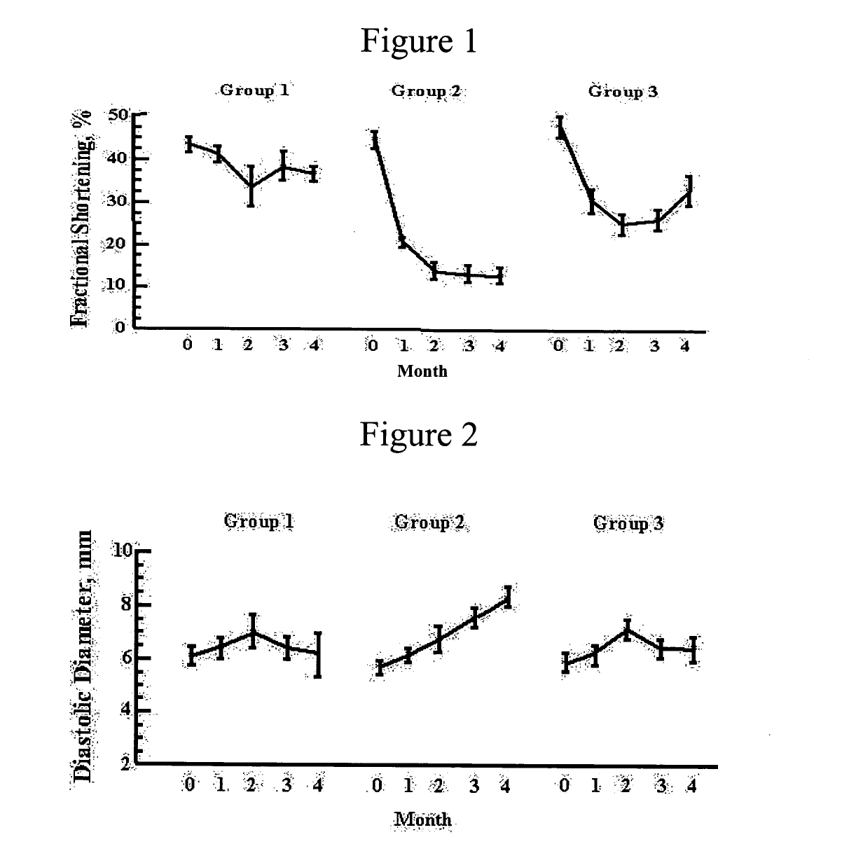

During the 4 months, the rats were physically active and had normal weight gain. There were no clinical signs of immunorejection in the HUCBC treated rats. FIG. 1 shows the echocardiographic left ventricular fractional shortening measurements [(LV Diastolic Diameter-LV Systolic Diameter)/LV Diastolic Diam.×100%] for each group of rats.

The left ventricular fractional shortening measurement for the Group 1 (normal) rats was 44+2%, but declined slightly with age. In Group 2 (infarction+transport medium), the LV fractional shortening measurement significantly decreased from the normal value by more than half to 20% at one month, and then further declined to a mean of 15+3% during the second through the fourth months after infarction. These measurements were identical to the fractional shortening measurements reported in rats with congestive heart failure (Litwin et al., 1994 Circ. 89;345-354; Sjaastad et al., 2000 J. Appl. Physiol. 89:1445-1454). In Group 3 (infarction+HUCBC), the fractional shortening measurement decreased to a mean of 31+3% at one month, 26+3% during the second and third month, and then increased to 34+3% at the fourth month. The Group 3 rat hearts contracted significantly better than the Group 2 rat hearts and the differences between Groups 3 and 2 were statistically significant (p<0.001). The left ventricles progressively dilated in Group 2 in contrast to ventricles in Group 3, which were normal in size or only slightly dilated in comparison with controls (FIG. 2).

The left ventricular anterior walls contracted significantly better in Group 3 than in Group 2 and were similar to the controls (Group 1) at the third and fourth months after infarction. In contrast, Group 2 anterior ventricular walls were severely hypokinetic between one and four months after infarction. The serial echocardiographic measurements in Group 1 and in Group 2 closely paralleled the reported echocardiographic changes in normal rats and infarcted rats (Litwin et al., 1994 Circ. 89;345-354).

### Example 2

HUCB Cells are Detectable in the Heart Tissue When Administered After Infarction Methods

HUCBC were fluorescently labeled with cholera toxin subunit B conjugated to fluorescein isothiocyanate prior to injection into the Group 3 rat hearts in order to facilitate microscopic cellular detection in the heart tissue.

Plasmids with hCMV IE promoter/enhancer driving green fluorescent protein (GFP) gene (5.7 kb) and the GenePORTER transfection reagent are used from Gene Therapy System (Chalfie et al., 1994 Science 263:802-805; Klein et al., 1997 Gene Therapy 4:1256-1260). HUCBC were plated on 100 mm dishes to obtain 60% confluence on the day of transfection. 8 μg of Plasmid GFP was added to each dish with a calcium phosphate precipitation method. After two days of GFP transfection, cultured HUCBC were carefully trypsinized, washed with PBS, and resuspended in Joklik modified medium at a density of 10⁶ cells/500 μl. Viability was assessed by Trypan Blue exclusion and was generally 80-90%. The transfection efficiency was greater than 85%. The labeled cells were injected into rat as described in Example 1. Fluorescence imaging of heart cells or tissue was performed with a Zeiss III fluorescent microscope and an Olympus IX70 confocal microscope.

### Results

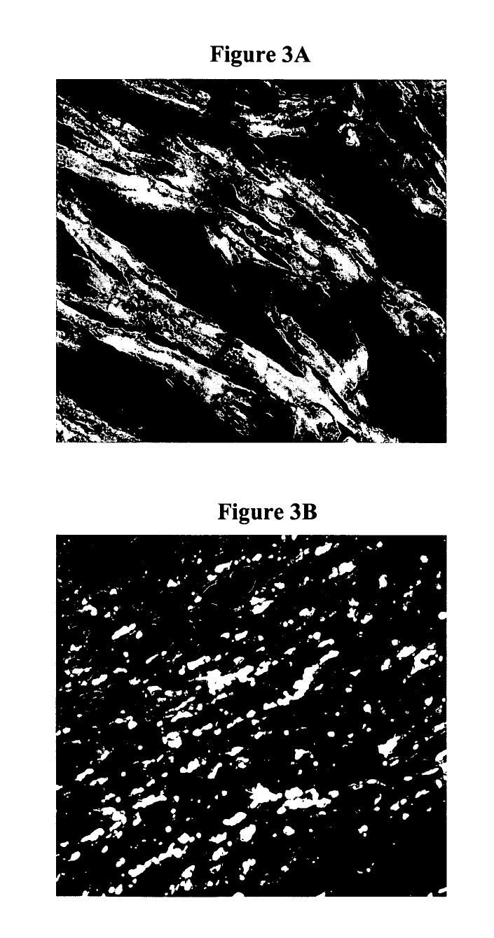

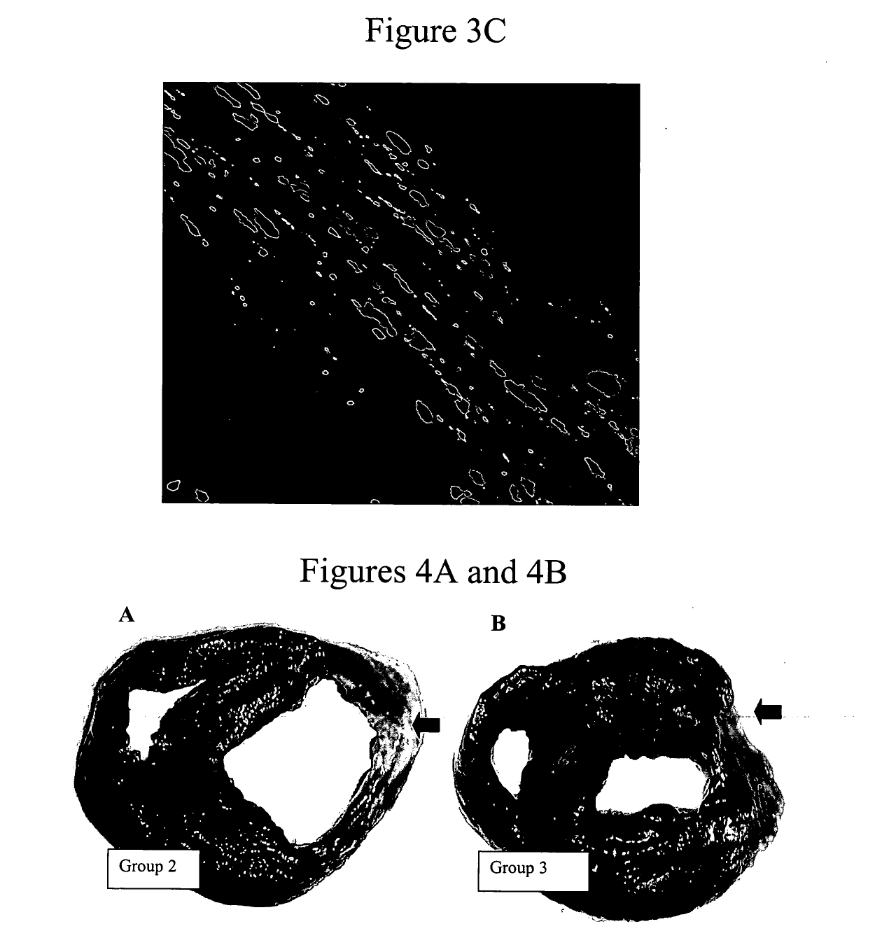

FIG. 3A shows normal heart tissue taken at the one month interval. FIG. 3B shows the typical appearance of the fluorescent HUCBC cells in the Group 3 heart tissue at one month after infarction. The HUCB cells were distributed in the heart muscle distal from the injection site and some cells have entered the zone of infarction. Aggregates of cells were also visualized. FIG. 3C shows the typical appearance of the HUCBC in Group 3 rat hearts at 4 months after infarction. At 4 months, the fluorescent HUCBC were aligned and in register with the host cardiomyocytes, which is consistent with the participation of the HUCB cells in a functional syncytium. Some of these cells had fusiform shapes. Little or no myocardial autofluorescence occurred in the Group 2 or in the Group 1, and there was no evidence of autofluorescence alignment with myocardial fibers in heart tissue from Group 2 or Group 1.

### Example 3

Administration of HUCB Improves Heart Function and Limits Size of Infarct

### Methods

Hematoxylin & Eosin staining was performed by immersing tissue slides in Harris Hematoxylin solution for 1 minute, rinsing, then immersing the slides in 1% aqueous Eosin Y for 2 minutes and rinsing. The slides were then dehydrated in ascending alcohol solutions (50%,70%, 95%×2, 100%×2) in Columbia stain jars. The slides were cleared with xylene and cover slipped. Tissue slices were examined for deformed nuclei, contraction bands, thinning and waviness of fibers, collagen fibers, interstitial hemorrhage, fibroblastosis and cellular infiltrates which are indicative of myocardial infarction. Tissue slices were also examined for lymphocytic infiltration and evidence of immunorejection.

The size of the infarct was determined by using tripenyl terazolium stain. The heart was sliced parallel to the atrioventricular sulcus from apex to base and six 0.3 cm thick slices are obtained. Each slice was rinsed in saline and incubated with 1% Tetrazolium at pH 7.4 and 37° C. for 15 minutes. Tetrazolium forms a red precipitate in the presence of intact dehydrogenase enzymes. Areas of myocardial necrosis lack dehydrogenase activity and therefore fail to stain (Fishbein et al., 1981 Am. Heart J. 101;593-600; Ytrehus et al., 1994 Am. J. Physiol. 267:H2383-H2390). The undamaged heart tissue turns a deep red color while the infarcted heart tissue turns light pink or white color as early as 30 minutes to 3 hours after acute coronary occlusion (Fishbein et al., 1981 Am. Heart J. 101;593-600; Adegboyega et al., 1997 Arch. Pathol. Lab. Med. 121:1063-1068). The heart slices were then fixed in 10% formalin. Tetrazolium has a diagnostic efficiency for myocardial infarction of 88% (Adegboyega et al., 1997 Arch. Pathol. Lab. Med. 121:1063-1068).

Each heart slice was photographed with a digital camera and the digital images transferred to the hard disk of a Pentium computer. The area of infarction and the area of normal ventricle were then determined by computer software analysis program (Image Pro) which permits determination of the area of the infarction and also the normal ventricle. The areas for each slice are then summed to calculate the total infarct area and the total area of the ventricles. Infarct size was expressed as infarct area divided by total ventricular muscle area. An investigator, unaware of the presence or absence of HUCBC in heart, determined all areas. Measurements were done in duplicate and results averaged. The standard deviation of the infarct measurements is ±5%.

### Results

Heart slices from Groups 1, 2, and 3 were stained with triphenyl tetrazolium chloride in order to quantitate myocardial infarction size. FIG. 4 shows horizontal sections taken through left and right ventricles of representative Group 2 and Group 3 rat hearts stained with tetrazolium. The Group 2 heart had a large densely scarred anterior wall, which encompassed 30% of the LV muscle mass and the dilated left ventricular cavity. In contrast, the anterior wall in the Group 3 heart was significantly less scarred, had areas of normal appearing myocardium, and encompassed only 9% of the LV muscle mass. In addition, the left ventricular cavity was not dilated in comparison with the Group 2 LV cavity. Quantitation of the scar area, total infarction size and total left ventricular muscle mass for each heart in each Group is currently in progress.

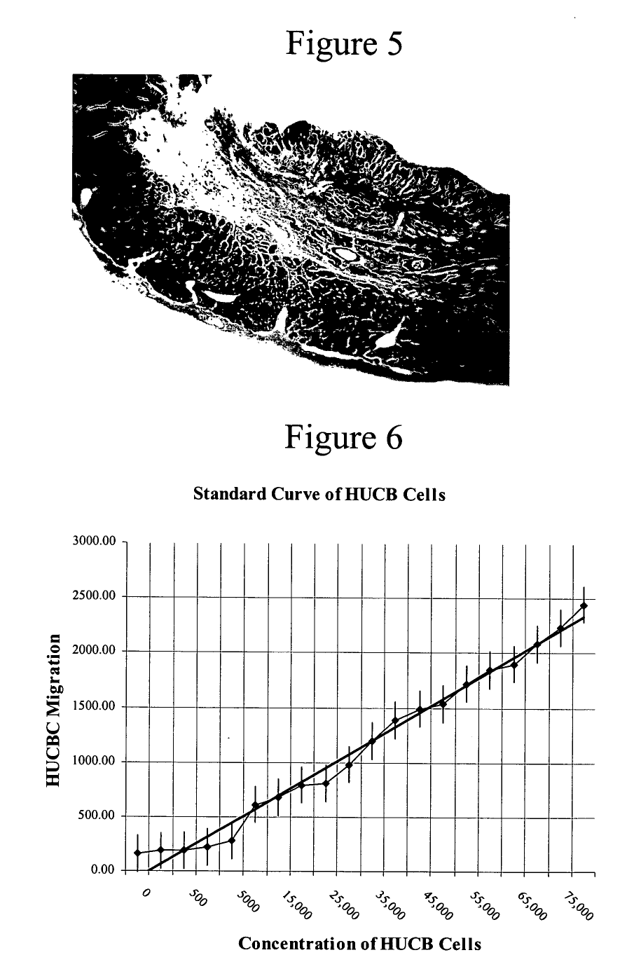

Hematoxylin & eosin and trichrome staining of myocardial tissue from Groups 1, 2 and 3 were performed at 1, 2, 3, and 4 months post-infarction, and were subsequently examined independently by a pathologist at the University of South Florida. There was no histological evidence of immunorejection in Group 3 hearts. FIG. 5 is a representative trichrome collagen stain from a different Group 2 and a Group 3 rat. Note the transmural infarction and large amounts of collagen in Group 2 but only subendocardial infarction in Group 3.

These preliminary studies strongly suggested that HUCB cells improve heart function, limit infarction size, but do not stimulate an immune rejection response in the host when injected into infarcted hearts. Additional studies may further define the role of HUCBC in treatment of acute myocardial infarction.

### Example 4

Determination of Optimal Time for Transplantation of HUCBC After Myocardial Infarction

A chemotactic assay, based on the migration of cells through a porous, inert polycarbonate micromembrane, is used to determine the optimal time for transplantation of HUCBCs.

### Methods

Rats are sacrificed at 1 hour, 3 hours, 6 hours, 12 hours, 24 hours, 48 hours, 96 hours and 192 hours after occlusion of the left anterior descending coronary artery. A normal control group is also used in which the animals do not undergo surgery. Tissue specimens, obtained from normal hearts and from the ischemic zone in each heart, are homogenized in Iscove's modified Dulbecco's medium (IMDM) (150 mg tissue per milliliter of IMDM), incubated on ice for 10 minutes, then centrifuged and kept on ice.

For this experiment, fresh normal heart tissue extract or tissue extracts (300 μL) prepared from the ischemic border of infarcted hearts at different times after occlusion of the left anterior descending coronary artery are placed in the lower chamber of 96 well micro-chemotaxis chamber (Coming) (Chen et al., 2001 Stroke 32:2682-2688; Muir et al., 1993 Anal. Biochem. 215:104-109; Sunder-Plassmann et al., 1996 Immunol. Invest. 24:49-63; Penno et al., 1997 Methods in Cell Science 19:189-195; Bignold, 1987 J. Immunol. Meth. 105:275-280; Xu et al., 1999 Hematology 4:345-356; Spessotto et al., Fluorescence assays to study cell adhesion and migration in vitro, In M Grant and C. Streuli (Eds.) Methods in Molecular Biology: The Extracellular Matrix. Humana Press, N.Y., 1998). In addition, serial dilutions of HUCBC at concentrations of 10⁶, 10⁵, 10⁴, and 10³ cells are placed in the first column of wells in the lower chamber for calibration purposes. No HUCBC are placed in the first column of wells in the upper chamber above these calibration cells. SDF-1, a chemoattractant that attracts HUCBC CD34+ cells in migration assays, is used as a positive control at a concentration of 100 ng/mL.

A 25×80 mm framed polycarbonate membrane with 5 μm pore size (NeuroProbe, Inc) is placed over the lower wells. Alternatively, the pore size may be increased or decreased. This membrane has a pore density of 4,000/mm², with a pore area of 19.365 μm², and a pore area/unit area of 7.85% (NeuroPore). GFP fluorescent-labeled HUCBC in Isolyte suspension are placed in the upper wells. Based on the density of the cells, the own experiments, and the stroke literature (Chen et al., 2001 Stroke 32:2682-2688; Muir et al., 1993 Anal. Biochem. 215:104-109; Sunder-Plassmann et al., 1996 Immunol. Invest. 24:49-63; Penno et al., 1997 Methods in Cell Science 19:189-195), 25 μl of fluorescent labeled HUCBC suspension (1×10⁶ cells/ml) is placed in the upper wells above the membrane. In addition, negative controls are set up in one column of wells in which the HUCBC are placed in upper wells but the corresponding bottom wells contain only tissue suspension media but no actual tissue chemoattractant in order to determine and correct for random migration (Muir et al., 1993 Anal. Biochem. 215:104-109; Sunder-Plassmann et al., 1996 Immunol. Invest. 24:49-63; Penno et al., 1997 Methods in Cell Science 19:189-195).

The migration chamber is placed in a water-jacket incubator at 37° C. with 5% CO₂. Migration of the HUCBC is allowed for a period that is long enough for many stimulated cells to migrate but not long enough for significant random migration or for many unstimulated cells to migrate at the negative control sites. The polycarbonate membrane and the lower chamber assembly are then centrifuged for 10 minutes at 400 g to force cells from the pores and the underside of the membrane into the wells in the lower chamber. The membrane is then read in the fluorescence reader to verify that the cells have been removed. The number of migrated cells into the lower wells is then determined by measuring the fluorescence of the cells in the lower well with a fluorescence microplate reader (BioTek Synergy). Measurements are done in triplicate and results are averaged. All experiments are done in triplicate to ensure reproducibility of the experiments. FIG. 6 shows the standard curve demonstrating the relationship between concentration of HUCBC in upper chamber and number of migrated cells in the lower chamber. The standard curve is established for each assay and for determining the sensitivity of the plate reader (BioTek,Inc).

### Results

Cell movement and migration is triggered by chemoattractant stimuli and by substrate-bound and soluble chemotactic agents and is a primary cellular process during embryonic development, the maintenance of a healthy adult, and the progression of conditions such as cancer. In the ischemic/infarcted myocardium paracrine growth factors and cytokines are released that attract various cell types into the injured heart that contribute to repair and ultimately healing of the infarcted tissue. For example, adult cardiomyocytes produce insulin-like growth factor, transforming growth factor-α, and heparin-binding epidermal growth factor-like growth factor (Toma et al., 2002 Circulation 105:93-98). The in-vitro chemotactic assay used for qualitative and quantitative analysis of this process in the ischemic/infarcted heart reveals the most optimal time after acute myocardial infarction to transplant HUCBC.

Additionally, the HUCBC-treated ischemic/infarcted myocardial tissue is examined for significant upregulated expression of natriuretic peptides, growth factors, and neovascularization factors that may limit edema formation, scar tissue formation, and improve heart blood flow and myocardial function.

### Example 5

Determination of Optimal Number of HUCB Cells for Treatment of Myocardial Infarction

Fluorescent labeled HUCBC are injected into infarcted hearts in amounts ranging from 0.5×10⁶ to 12×10⁶ cells. Thereafter, heart function is determined at 1 week and 1, 2, 3, 4 months by echocardiographic measurements and infarct size is determined by tetrazolium staining and by hematoxylin & eosin staining.

### Methods

Rats are randomized to fluorescent labeled HUCBC numbers of 0 (controls), 0.5×10⁶, 1×10⁶, 3×10⁶, 6×10⁶ and 12×10⁶ that are directly injected into the periphery of the infarct of each rat. The optimal time for the injection of HUCBC is determined from Example 4. Infarct size is determined by tetrazolium staining in a subset of rats at each time interval. For purposes of the analysis, viable myocardium is tissue that stains red whereas non-viable infarcted myocardium stains pink or white. The area of infarction and the area of normal ventricle in each heart slice are determined by computer analysis (Image Pro). The areas in each slice are summed and the percent infarction is then expressed as the total area of infarction/total area of the ventricles. Infarct size is also quantitated in some rat ventricles by hematoxylin and eosin and trichrome staining. Collagen density is then measured in 10 microscopic fields having the highest infarct HUCBC grafted cell density at 20× magnification and compared with the collagen density from 10 random fields in nongrafted infarcts. HUCBC location, orientation, and morphology are determined by microscopy at each interval with Pontamine Sky Blue and also NUMA stains.

Pontamine Sky Blue Stain Methodology for Host Cardiomyocytes is performed on serial 6 μm frozen sections prepared with a cryostat microtome (Bright 5030). 10 mL of phosphate buffered saline (PBS) is mixed with 100 μL of DMSO and 10 μg of Pontamine sky blue powder. The mixture is applied to the heart tissue sections on glass slides for 30 minutes, washed off with PBS, and the slides are dipped in 100% alcohol and then washed with PBS. The slides are then placed in xylene and subsequently covered. The Pontamine Sky Blue stains the heart tissue but does not stain the fluorescent HUCBC.

Human Nuclear Matrix Antigen (NUMA) staining methodology is used to identify the Nuclei of HUCBC. Six μm heart tissue sections are treated with H₂O₂ for 15 minutes, washed 3 times for 10 minutes each with PBS, then treated with PBS (870 μl), 10× Triton (30 μl), and 10% goat serum (100 μl) for 60 minutes. The slides are incubated overnight at 4° C. with NUMA 1:200, PBS (950 μl), 10× Triton (30 μl), and 2% goat serum (20 μl). On day 2, the slides are washed 3 times with PBS. Goat anti-mouse antibody (2 μl/ml), PBS (970 μl), 10× Triton (30 μl), and 2% goat serum (20 μl) are then added for 120 minutes. The slides are washed with PBS, treated with ABC kit for 60 minutes, treated with DAB buffer solution (9:1 ratio) for 15 minutes, then washed and dried. NUMA stains human but not rat nuclei in heart tissue.

Transthoracic echocardiography is performed on all animals by a sonographer who is unaware of the rats' category/treatment. Echocardiographic and Doppler tissue imaging measurements are made at 1 week and 1, 2, 3, and 4 months post coronary artery occlusion (Sahn et al., 1978 Circ. 58:1072-1083). This monitoring technique is accurate and reproducible in normal rats and rats with infarction (Litwin et al., 1994 Circ. 89:345-354; Burrell et al., 1996 Clin. Exp. Pharm. Phy. 23:570-572). A commercially available echocardiographic system (Siemens/Acuson, Mountain View CA) is used that is equipped with a 15 MHz ultrasound transducer. This equipment has a resolution to 0.1 mm (Litwin et al., 1994 Circ. 89:345-354; Burrell et al., 1996 Clin. Exp. Pharm. Phy. 23:570-572). Rats are anesthetized with ketamine and xylazine and examined in the left lateral position. Short axis and long axis two dimensional and M mode echocardiograms are obtained at the level of the papillary muscles and are recorded at a paper speed of 100 mm/s. The long axis view is used to guide perpendicular angulation of the transducer to acquire short axis views. All images are stored on videotapes. Three to 5 cardiac cycles are measured and the values averaged for each rat.

A physician and a sonagrapher expert in echocardiography, who are unaware of the rats' category, analyze the echocardiographic images. Measurements of anteroseptal end-diastolic wall thickness, posterior end-diastolic wall thickness, and LV internal dimensions at end diastole (LVDD) and end-systole (LVSD) are made according to the American Society for Echocardiography leading-edge method (73). Fractional shortening (FS) of the LV is calculated using the formula: FS=(LVDD-LV SD)/LVDD×100%. The independent observer scores the wall motion of the anterior septum and anterior apical and posterior lateral and posterior apical walls where 1=normal/hyperkinetc; 2=hypokinetic; 3=akinetic; 4=dyskinetic; 5=aneurysmal motion. In addition, the wall thinning ratio (anterior wall thickness/posterior wall thickness) and relative wall thickness (2×posterior wall thickness/LV internal dimension) are measured to determine if changes in wall thickness and cavity size are proportionate or disproportionate.

A 4 MHz transducer pulsed-wave and M mode is used for Doppler tissue imaging (DTI) of the septum and lateral-posterior walls. From the DTI tracings, the peak velocity of (1) isometric contraction (2) systolic excursion (3) isometric relaxation and (4) early and late ventricular diastolic excursion are measured. Five beats are averaged for each measurement. The variation of myocardial velocity after coronary occlusion is then expressed as a percentage of the baseline velocity measurement (Derumeaus et al., 1998 Circ. 97:1970-1977; Gorcsan et al., 1997 Circ. 95:2423-2433).

The results are expressed as the mean±standard error of the mean. Some experiments are repeated at random to ensure reproducibility of the data. The difference between two groups is tested by the Student's t test. Differences among more than two groups are tested by analysis of variance (ANOVA). Multiple comparisons between groups within any one group are performed with a Bonferroni modification of the t test. A value of p<0.05 is judged significant.

### Results

Statistical correlations are determined between the number of HUCBC transplanted and infarct size, ventricular fractional shortening, wall thinning ratio, relative wall thickness, and ventricular compliance as determined by isometric relaxation and ventricular diastolic excursion. It is expected that an increase in HUCBC cell number will decrease infarct size and increase ventricular function but that the changes in these measurements will eventually reach a plateau despite further increases in HUCBC cell number. A minimum of 7 to 9 rats per cell dose are required for a study with a p value=0.05 and a 90% chance of detecting a 20% difference between groups.

If significant ventricular hypertrophy occurs that may confound measurements of infarct area/ventricular area, then the number of control infarctions will be substantially increased and noninfarcted and infracted areas compared separately between each group and reported separately. Cyclosporin, at approximately 15 mg/kg/d, is utilized if clinical or histological signs of cellular rejection are detected. Because tetrazolium is light sensitive, all staining is done in a darkened room. Tissue handling and staining techniques in this project do not permit detailed examination of individual HUCBC for cardiac specific protein, and therefore, HUCBC are examined for cardiomyocyte specific proteins as described in Example 6.

If no differences in infarct size are detected or if significant non-infarct area hypertrophy occurs compared controls, tissue samples will be examined for neovascularization by examining HUCBC fluorescent cells for endothelial cell markers: the VEGF receptor Flt-I, von Willebrand factor and Factor VIII (Murry et al., 1996 J. Clin. Invest. 98:2512-2523). Tissue is also examined for increased expression of natriuretic peptides & growth factors (insulin-like growth factor, transforming growth factor-α, heparin-binding epidermal growth factor).

### Example 6

Characterization of HUCBC-Derived Cells After Administration

In order for transplanted HUCBC to limit damage from acute myocardial infarction, the cells must divide, develop into cardiomyocytes, and induce neovascularization. In this project it is determined whether fluorescent labeled HUCBC transplanted into infarcted rat ventricles divide, express the cardiac specific proteins (α-actinin and β-myosin heavy chain protein found in fetal myocytes and express troponin I, which is present in fetal and adult myocytes, and express endothelial cell markers normally present in blood vessels.

Fluorescent HUCBC are transplanted into infarcted rat ventricles as described in Examples 1-5. The optimal time and the optimal number of HUCBC for injection each ventricle are determined as described in Examples 4 and 5. In group 1 rats, HUCBC+Isolyte media are injected into the ischemic border zone of the infracted rat ventricle. In group 2 rats, only Isolyte media is injected into the ischemic zone that borders on the infarction. Group 3 rats serve as controls to permit documentation of changes in heart tissue that occur with increased rat age and weight. In addition, the second and third groups permit documentation of autofluoresence, nonspecific staining, staining of fibroblasts and necrotic tissue and also permit determination of the sensitivity and specificity of the staining techniques.

When determining the proliferation of transplanted HUCBC, a subset of group one animals is sacrificed at 2 to 7 days. The hearts are subjected to 0.07% collagenase perfusion for isolation of all myocytes, which is routinely performed (Henning et al., 2000 J. Cardiovasc. Pharmacol. Therapeut. 5(4):313-322). Fluorescent positive cells are then counted and characterized by flow cytometry and the cell counts confirmed by fluorescent microscopy (Min et al., 2002 J. Applied Physiology 92:288-296).

When characterizing the presence of cardiac specific proteins and endothelial cell markers, groups 1-3 rats are followed for four months. This time is based on the expression of cardiac and endothelial specific proteins by bone marrow and embryonic cells in infarcted ventricles (Min et al., 2002 J Applied Physiology 92:288-296; Murry et al., 1996 J. Clin. Invest. 98:2512-2523; Toma et al., 2002 Circ. 105:93-98; Volk & Geiger, 1984 EMBO 3:2249-2260) and also permits determination of stability of new cellular proteins over time. A subset from each group is sacrificed at 1 week, and 1, 2, 3, and 4 months.

Cardiac Muscle Protein Immunohistochemistry is performed on tissue slides. Immunofluorescent techniques are used to identify α-actinin protein and β-myosin heavy chain protein in transplanted HUCBC. α-actinin protein and β-myosin heavy chain protein are present in fetal but not in adult cardiomyocytes or HUCBC. In addition, cardiac troponin I protein, which is present in fetal and adult myocytes but not HUCBC, is determined in GFP positive HUCBC. Primary monoclonal antibodies are used to detect muscle 60 -actinin and β-myosin heavy chain (Toma et al., 2002 Circ. 105:93-98; Min et al., 2002 J Applied Physiology 92:288-296; Reinecke et al., 1999 Circ. 100:193-202). A fluorescent secondary antibody (TRITC-conjugated goat anti-mouse IgG) is used for 45 minutes. Cardiac troponin I staining is performed with a goat polyclonal IgG anti-cTnI antibody, then with a rabbit anti-goat conjugated rhodamine IgG antibody (Toma et al., 2002 Circ. 105:93-98; Min et al., 2002 J Applied Physiology 92:288-296; Reinecke et al., 1999 Circ. 100:193-202). HUCBC labeled with GFP are then identified in heart tissue and these cells are examined for α-actinin, β-myosin heavy chain, troponin I, and sarcomeric organization of contractile proteins is examined with a Zeiss fluorescent microscope and an Olympus confocal microscope.

In addition, immunofluorescent techniques are used to identify the VEGF endothelial cell receptor Flt-1 involved in endothelial cell growth, Von Willebrand factor, and Factor VIII, which are present in endothelial cells but not HUCBC. Monoclonal antibodies are to Flt-1, Von Willebrand factor, and Factor VIII are used, and are followed by a FITC conjugated secondary antibody (Gorcsan et al., 1997 Circ. 95:2423-2433). Additionally, the number of capillary vessels in the scar tissue of all groups is determined using light microscopy (×400 magnification). Five high-powered fields in each scar are randomly selected, and the number of capillaries in each is averaged and expressed as the number of capillary vessels per high power field and compared with group 2 and group 3 rats.

### Results

It is expected that HUCBC will proliferate within 72 hours of transplantation and that the cell count will significantly exceed the number initially transplanted. It is also expected that fluorescent cells will express specific cardiac proteins and endothelial cell markers within 1 to 2 months of transplantation and that the number of capillaries in the HUCBC treated rats will exceed group two and three. A minimum of 10-20 rats in each group is required to achieve a ≧60% chance of detecting cardiac and endothelial specific proteins in the HUCBC with 5-10% variability in the staining. Some experiments are repeated at random to ensure reproducibility.

If the fluorescent marker fades or is poorly transmitted in dividing cells, cholera toxin subunit B conjugated to fluorescein will be used because this marker persists in HUCBC for ≧6 months in the studies. If difficulty is experienced counting fluorescent cells, expression of Ki67 in HUCBC will be measured in nuclei using an anti-mouse Ki67 antibody. Ki67 is expressed in cycling cells in G1, S, G2 and early mitosis and is used to demonstrate cell proliferation (Orlic et al., 2001 Nature 410:70-705). If difficultly is experienced demonstrating cardiac proteins in large tissue sections, then single ventricular myocardial cells will be isolated by collagenase perfusion of the intact heart. Fluorescent positive cells will be isolated and immunostained for (α-actinin, β-myosin, troponin I, and endothelial cell markers.

If specific cardiac and endothelial proteins are not identified, HUCBC treated ischemic myocardial tissue will be examined for increased expression of natriuretic peptides (atrial natriuretic peptide and brain natriuretic peptide) that can limit edema formation in the heart, growth factors (insulin-like growth factor, transforming growth factor-β, heparin-binding epidermal growth factor) that can cause hypertrophy and remodeling, and neovascularization factors (vascular endothelial growth factor, fibroblastic growth factor).

We claim:

1. A method of treating a circulatory disorder, comprising: administering an effective amount of a composition comprising an umbilical cord blood cell to an individual with a circulatory disorder.

2. The method of claim 1, wherein the umbilical cord blood cell differentiates into a cardiac muscle cell.

3. The method of claim 1, wherein the circulatory disorder is selected from the group consisting of cardiomyopathy, myocardial infarction, and congenital heart disease.

4. The method of claim 1, wherein the circulatory disorder is a myocardial infarction.

5. The method of claim 4, wherein the umbilical cord blood cell is administered within approximately 48 hours after the onset of the myocardial infarction.

6. The method of claim 1, wherein the individual is a human.

7. The method of claim 1, wherein the umbilical cord blood cell is a human umbilical cord blood cell.

8. The method of claim 7, wherein the human umbilical cord blood cell is a mesenchymal cell.

9. The method of claim 1, wherein the umbilical cord blood cell is administered directly to heart tissue.

10. The method of claim 1, wherein the umbilical cord blood cell is administered systemically.

11. The method of claim 1, wherein the umbilical cord blood composition comprises at least about 6 million white blood cells per milliliter.

12. A method for treating myocardial infarction, comprising administering a composition comprising an human umbilical cord blood cell to an individual having a myocardial infarction in an effective amount sufficient to produce cardiac muscle cells in the heart of the individual, wherein the umbilical cord blood cell differentiates into a cardiac muscle cell.

13. The method of claim 12, wherein the differentiation into a cardiac muscle cell treats myocardial infarction by reducing the size of the scar resulting from the myocardial infarction.

14. The method of claim 12, wherein the umbilical cord blood cell is administered directly to the heart of the individual.

15. The method of claim 12, wherein the umbilical cord blood cell is administered systemically.

16. The method of claim 12, wherein the human umbilical cord blood cell is a mesenchymal cell.

17. The method of claim 12, wherein the umbilical cord blood cell is administered within approximately 48 hours after the onset of the myocardial infarction.

18. The method of claim 12, wherein the composition comprises at least about 6 million white blood cells per milliliter.

19. A method of treating an injured tissue in an individual comprising:
(a) determining a site of tissue injury in the individual; and
(b) administering an umbilical cord blood composition into and around the site of tissue injury, wherein the umbilical cord blood composition comprises a cell that differentiates into a cardiac muscle cell after administration.

20. The method of claim 19, wherein the tissue is cardiac muscle.

21. The method of claim 20, wherein the umbilical cord blood composition comprises a mononuclear cell fraction isolated from human umbilical cord blood; plasma or fetal bovine serum; and DMSO.

22. The method of claim 20, wherein the tissue injury is a myocardial infarction.

23. The method of claim 22, wherein the differentiation into a cardiac muscle cell treats myocardial infarction by reducing the size of the scar resulting from the myocardial infarct.

24. The method of claim 22, wherein the umbilical cord blood cell is administered within approximately 48 hours after the onset of the myocardial infarction.

25. The method of claim 19, wherein the umbilical cord blood composition is prepared by the steps comprising:
(a) obtaining whole cord blood from a neonatal umbilical cord;
(b) enriching the cord blood for mononuclear cells; and
(c) resuspending the cord blood enriched for mononuclear cells with plasma or fetal bovine serum, and DMSO.

26. The method of claim 25, wherein the umbilical cord blood composition comprises at least about 6 white blood cells per milliliter.
